(19) United States
(12) Patent Application Publication
(10) Pub. No.: US 2025/0143640 A1
(43) Pub. Date: May 8, 2025

(54) SENSOR ARRANGEMENTS FOR A SENSING GARMENT

(71) Applicant: Siren Care, Inc., San Francisco, CA (US)

(72) Inventors: Jie FU, Shanghai (CN); Dan Dan Miao, San Francisco, CA (US)

(21) Appl. No.: 18/766,285

(22) Filed: Jul. 8, 2024

Related U.S. Application Data
(63) Continuation of application No. 18/518,231, filed on Nov. 22, 2023, now abandoned.
Application No. 18/518,231 is a continuation of application No. PCT/US2022/031192, filed on May 26, 2022.
(60) Provisional application No. 63/194,144, filed on May 27, 2021.

(51) Int. Cl. A61B 5/00; A61B 5/01

(52) U.S. Cl. CPC A61B 5/6807; A61B 5/01; A61B 2562/12

(57) ABSTRACT

Sensor arrangements for a user monitoring system and methods of making thereof are described herein. In one variation, the method comprises providing an interconnected array of bases. For each base, the method includes applying a conductive material to a recessed portion in the base to form at least one conductive region. At least one sensor can be coupled to the at least one conductive region. A conductive member can be coupled to the at least one conductive region. An encapsulating material can be dispensed onto the base, thereby encapsulating the at least one sensor and at least a portion of the conductive member in the recessed portion.

### CROSS-REFERENCE TO RELATED APPLICATIONS

This application is a continuation of U.S. patent application Ser. No. 18/518,231, filed Nov. 22, 2023, which is a continuation of International Application No. PCT/US2022/031192, filed May 26, 2022, which claims the benefit of U.S. Provisional Patent Application No. 63/194,144, filed May 27, 2021, each of which is incorporated by reference herein in its entirety for all purposes.

### TECHNICAL FIELD

This invention relates generally to the field of health care and more specifically to new and useful sensor arrangements for sensing garments and methods for making the same.

### BACKGROUND

Constant monitoring of one or more body parts can help with early detection of infections. For instance, constant monitoring of a diabetic patient's feet can prevent foot ulcers from progressing into gangrene. Therefore, it has become increasingly common for healthcare practitioners to recommend self-care plans including at-home monitoring to their patients.

However, reliable and easy-to-use monitoring systems that provide a complete and accurate picture of the health of one or more body parts are hard to find. For example, readily available handheld monitoring systems are severely restrictive. Such monitoring systems are configured to monitor one location at a time rather than monitoring a complete body part.

Therefore, there is a need for new and improved monitoring systems that can monitor a suitable body part in a holistic manner. Furthermore, there is a need for new and improved methods for making such monitoring systems in a scalable and reliable manner.

### SUMMARY

Generally, in some variations, a method for making a sensor arrangement for a user monitoring system may include providing an interconnected array of bases, and for each base, applying a conductive material to a recessed portion in the base to form at least one conductive region, coupling at least one sensor to the at least one conductive region, coupling a conductive member to the at least one conductive region, and dispensing an encapsulating material onto the base, thereby encapsulating the at least one sensor and at least a portion of the conductive member in the recessed portion. Furthermore, in some variations, a sensor arrangement may include at least one base comprising a recessed portion having at least one conductive region; and at least one conductive member coupled to the at least one base, wherein at least one sensor is coupled to the at least one conductive region, wherein a portion of the at least one conductive member in coupled to the at least one conductive region, and wherein an encapsulating material encapsulates the at least one sensor and the portion of the at least one conductive member in the recessed portion of the at least one base.

### BRIEF DESCRIPTION OF THE DRAWINGS

FIG. 1 is an illustrative schematic of an exemplary variation of a user monitoring system.

FIG. 2 illustrates an exemplary variation of a base.

FIG. 3 is an exemplary variation of a base illustrating a portion of a conductive member coupled to the base.

FIG. 4 is an exemplary variation of a base illustrating an encapsulating material encapsulating a sensor and a portion of a conductive member within the base.

FIG. 5 is an exemplary variation of an interconnected array of bases.

FIG. 6 is a flowchart of an exemplary variation of a method of making a sensor module.

FIG. 7 is an exemplary variation of an exploded view of a sensor module being coupled to a garment.

FIG. 8 is an exemplary variation of a method of making a sensor arrangement.

FIG. 9 is an exemplary variation of a sensor arrangement illustrating the sensor arrangement sealed in a protective film.

FIG. 10 is an exemplary variation of an example method of making a sensor arrangement for a user monitoring system.

### DETAILED DESCRIPTION

Non-limiting examples of various aspects and variations of the invention are described herein and illustrated in the accompanying drawings.

Systems and methods for monitoring a user and methods for making a user monitoring system are described herein. More specifically, a sensor arrangement to monitor a user (e.g., monitor a foot of a user) and methods of making such a sensor arrangement are described herein.

Foot problems are common in people with diabetes. Without early detection and treatment, small sores and blisters on the foot can transform into severe infections such as gangrene in patients with diabetes. Therefore, constant and consistent monitoring of the feet is often recommended for such patients.

A traditional method of monitoring the feet is visual inspection. However, this has several limitations. For example, small blisters and sores may go unnoticed upon visual inspection. Additionally, a nascent sore or blister that was undetected during visual inspection can worsen by the time a subsequent inspection is conducted.

A different method of monitoring the feet for infections comprises collecting data (e.g., temperature data, pressure data, moisture data, a combination thereof, and/or the like) from various regions of a foot of a patient. However, for early and accurate detection of infections, the collection of data has to be continuous. For example, using a handheld device, such as thermometer, at various locations of the foot may not provide accurate information. Instead, such a handheld device would capture the temperature of a specific location of a foot at a specific time. It is possible that at that particular time, the captured reading may show a normal temperature reading (e.g., no sore and/or a sore at a very nascent stage). However, as time progresses an infection may go undetected (e.g., a new sore and/or a nascent sore worsening) until another reading is taken, which may be after several hours or several days.

In contrast, continuous monitoring of the feet can drastically improve home care and early detection of diabetic foot conditions. For instance, continuous monitoring facilitates assessment of temperature, pressure, moisture, and/or the like over longer periods of time. Readings over longer periods of time can be used to produce patterns (e.g., patterns of temperature over the course of an hour or through the day) for a patient. These patterns can be taken into consideration when assessing foot conditions in the patient. Such an analysis of patterns (e.g., temperature patterns) that are specific to the patient can provide for more accurate assessments. Patterns from various locations of the foot can further improve monitoring of the foot. Put differently, generating patterns for different regions of the foot by collecting sensor data (e.g., temperature data, pressure data, etc.) from various regions of the foot simultaneously can further improve monitoring of the foot for early detection Exemplary methods for monitoring of foot for early detection of inflammation from the user monitoring system using sensor data (e.g., temperature data) are described in U.S. Patent App. Pub. No. 2017/0188841, which is hereby incorporated in its entirety by this reference.

A user monitoring system with a sensor arrangement that is configured to collect sensor data from various locations of the foot simultaneously in a continuous and reliable manner is described herein. Since the sensor arrangement may be incorporated into a garment that is in contact with the foot of the patient for long periods of time, in some variations, the sensor arrangement may be designed factoring in the comfort of the patient. Furthermore, in some variations, the sensor arrangement can be designed to be robust and less prone to failure since they may be integrated into a garment and/or footwear that is worn by patients daily.

Additionally, methods of making robust sensor arrangements for a user monitoring system in a scalable and reliable manner is described herein. In some variations, such methods may reproduce reliable and consistent sensors and consequently sensor arrangements. In some variations, such methods may be scalable and can reproduce multiple sensor arrangements for various user monitoring systems.

### User Monitoring System

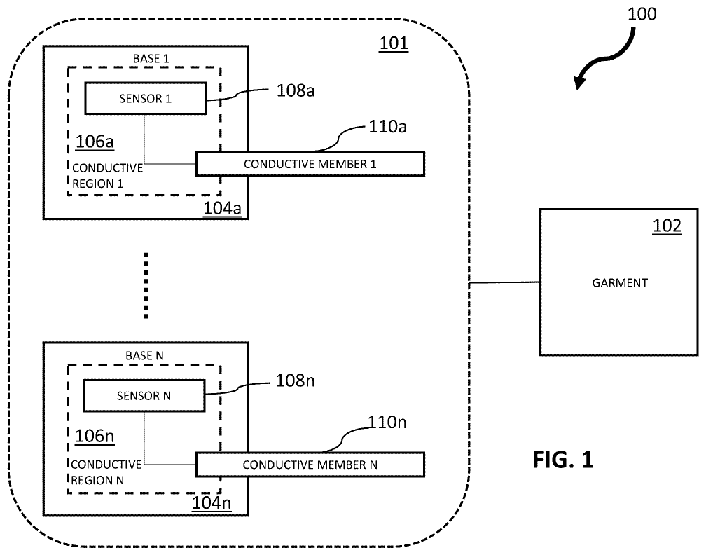

FIG. 1 is an illustrative schematic of an exemplary variation of a user monitoring system 100. The user monitoring system 100 can include a sensor arrangement 101. The sensor arrangement 101 can include one or more bases 104 (e.g., bases 104a-104n) each coupled to at least one respective conductive member 110 (e.g., conductive members 110a-110n). For instance, in FIG. 1, base 104a is coupled to conductive member 110a and base 104n is coupled to conductive member 110n.

Each base 104 can include at least one sensor 108 (e.g., sensors 108a-108n) that can be coupled to a conductive region 106 (e.g., conductive regions 106a-106n) within that base 104. For example, in FIG. 1, sensor 108a is coupled to conductive region 106a and sensor 108n is coupled to conductive region 106n.

A base 104 can be coupled to the conductive member 110 via the conductive region 106. In some variations, the sensor 108 included in the base 104 can be coupled to the conductive member 110 via the conductive region 106. For example, in FIG. 1, conductive member 110a is coupled to the sensor 108a via the conductive region 106a. In a similar manner, conductive member 110n is coupled to the sensor 108n via the conductive region 106n.

FIG. 1 shows two bases 104a and 104n and two conductive members 110a and 110n solely for illustrative purposes. It should be readily understood that the sensor arrangement 101 can include any number of conductive members 110 coupled to one or more bases 104. For instance, the sensor arrangement 101 may include a single base 104 coupled to a single conductive member 110 (e.g., only base 104a coupled to conductive member 110a without base 104n or conductive member 110n in FIG. 1). Similarly, the sensor arrangement 101 can include a third conductive member 110b (not shown in FIG. 1) coupled to a third base 104b (not shown in FIG. 1).

In some variations, each of the conductive members 110 coupled to the base 104 (e.g., via conductive region 106) can be bundled together to form the sensor arrangement 101, as further described below. The sensor arrangement 101 can be coupled to a garment 102 configured to be placed on a foot of a user (e.g., a patient with diabetes).

### Garment

The garment 102 can provide a substrate or platform for the sensor arrangement 101. In some variations, the garment 102 includes a sock configured to be placed or worn on a foot of the user. However, the garment 102 may alternatively be any suitable component to be positioned on the foot such as a shoe, a slipper, an insole, etc. For example, in some variations, a garment 102 may be a shoe or shoe component configured to house the sensor arrangement 101. For example, the shoe may include an insole configured to receive one or more sensor arrangements 101. The insole may include one or more sensor arrangements 101 coupled to the insole by a cover. The garment 102 may be configured specifically for a left foot (e.g., include a toe box accommodating contours of a left foot), for a right foot (e.g., include a toe box accommodating contours of a right foot), or may be universally or suitable for both feet. The garment 102 may include one or more labels that are sewn, woven, or otherwise incorporated into or coupled to the garment. Examples of labels include an indication of left or right foot compatibility, size (e.g., small, medium, large, or numeric size), or other identifying info.

In some variations, the garment 102 can be any suitable garment (e.g., pants, short pants, tights, leggings, leg warmer, shirt, arm warmer, glove, mitten, scarf, hat, headband, chest band, etc.) configured to be placed adjacent to one or more suitable body parts of the user for monitoring. In some variations, the garment 102 may include a flexible material such as a textile (e.g., polyester, cotton, etc.) that is configured to conform to the foot or other body part of the user. In some variations, the garment 102 may include one or more recessed portions to receive at least a portion of the sensor arrangement 101, as further described below.

In some variations, the garment 102 may further include at least one housing. The housing may include at least one cavity for receiving the sensor arrangement 101. The housing may be positioned on any suitable portion of the garment 102. In some variations, the housing may be secured to the garment 102 by being enclosed between the garment 102 and a cover.

In some variations, the garment 102 can include various components for operating the user monitoring system 100. For example, the housing included in the garment 102 can house these various components. In one variation, the housing can include at least one processor (e.g., CPU), at least one memory device (which can include one or more computer-readable storage mediums), at least one communication module, and at least one power source. In some variations, the housing may further include one or more additional activity or other sensors (e.g., an accelerometer, a gyroscope, or an inertial measurement unit). One or more of these components may be arranged on one or more electronic circuit boards (e.g., PCBA), which in turn be mounted to the housing.

The processor and memory device may cooperate to provide a controller for operating the user monitoring system 100. For example, the processor may receive sensor data from one or more sensors, and the sensor data may be stored in one or more memory devices. In some variations, the processor and memory may be implemented on a single chip, while in other variations they can be implemented on separate chips.

The controller can operate in an inactive state and in an active state. The controller may, for example, toggle between the inactive state and the active state based on user input (e.g., pressing of a button) and/or sensor data (e.g., from activity sensors, processing of temperature data, etc.) suggesting placement of the garment 102 on a user. In the inactive state, the controller may be in a “sleep” mode (e.g., to conserve energy in the power source). In the active state, the controller may be in an “awake” mode in which sensor data is received, processed, and/or stored in the memory device for use in monitoring. In some variations, the controller may be similar to the controller described in U.S. Patent Pub. No. 20170188841, incorporated by reference above.

The communication module may be configured to communicate sensor data and/or other information to an external computing device. The external computing device may be, for example, a mobile computing device (e.g., mobile telephone, tablet, smart watch), laptop, desktop, or other suitable computing device. The external computing device may be executing a native application for presenting sensor data (and/or the results of analysis thereof) through a user interface to a user. Additionally or alternatively, the communication module may be configured to communicate to one or more networked devices, such as a hub paired with the system, a server, a cloud network, etc.

The communication module may communicate via a wired connection (e.g., including a physical connection such as a cable with a suitable connection interface such as USB, mini-USB, etc.) or a wireless network (e.g., through NFC, Bluetooth, WiFi, RFID, or any type of digital network that is not connected by cables). For example, devices may directly communicate with each other in pairwise connection (1:1 relationship), or in a hub-spoke or broadcasting connection (“one to many” or 1:m relationship). As another example, the devices may communicate with each other through mesh networking connections (e.g., “many to many”, or m:m relationships), such as through Bluetooth mesh networking. Wireless communication may use any of a plurality of communication standards, protocols, and technologies, including but not limited to, Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (WiFi) (e.g., IEEE 802.11a, IEEE 802.11b, IEEE 802.11g, IEEE 802.11n, and the like), or any other suitable communication protocol. Some wireless network deployments may combine networks from multiple cellular networks or use a mix of cellular, Wi-Fi, and satellite communication.

Additionally, the housing may include one or more power sources, which may function to provide electrical power to the processor, communication module, sensors, and/or any other electrical components. For example, the power source may include one or more batteries. In some variations, the power source may be rechargeable such as through wireless charging methods (e.g., inductive charging, RF coupling, etc.) or by harnessing kinetic energy such as that generated through motion (e.g., when the user walks while wearing the garment 102).

In some variations, the garment 102 may be similar to any one or more garments described in U.S. patent application Ser. No. 16/836,800, the content of which is hereby incorporated by reference in its entirety.

### Cover

In one variation, the user monitoring system 100 may further include at least one cover (not shown in FIG. 1). The cover can be coupled to the garment 102 such that the sensor arrangement 101 is located between the garment 102 and the cover. The cover may be coupled to an internal surface of the garment 102, over a sensor arrangement 101 positioned on the inner surface of the garment 102. Alternatively, the cover may be coupled to an external surface of the garment 102, over a sensor arrangement 101 on the outer surface of the garment.

In some variations, the cover may include a single piece. Alternatively, the cover may include multiple pieces that collectively enclose the sensor arrangement 101 over the garment 102. In one variation, the cover may have any suitable shape and size larger than the outline of the sensor arrangement 101. In other variations, the cover may be oval, circular, or other suitable shape.

The cover may include any of various suitable materials. For example, the cover may include a textile (e.g., polyester, cotton, etc.) that is cut to a desired shape. In some variations, such a textile cover may be lined with a material suitable for heat sealing the cover to the garment 102, such as a thermoplastic material (e.g., thermoplastic polyurethane). Additionally or alternatively, the textile cover may be coupled to the garment 102 with epoxy or other adhesive, sutures, and/or fasteners (e.g., rivets). In other variations, the cover may include an epoxy or other adhesive (e.g., silicone glue), or sewn threads (e.g., embroidery) that directly secures the sensor arrangement 101 to the garment 102.

In some variations, the cover may be similar to any one or more covers described in U.S. patent application Ser. No. 16/836,800, the content of which is hereby incorporated by reference in its entirety.

### Sensor Arrangement

In some variations, the sensor arrangement 101 can be positioned on the garment 102 (e.g., an internal surface of the garment 102). The sensor arrangement 101 can collect sensor data (e.g., skin temperature measurements of the user, foot pressure measurements of the user, etc.) from one or more various locations of at least one suitable part of the body of the user, when the garment 102 is placed on the body part of the user.

In some variations, the conductive members 110 coupled to the bases 104 can be grouped together (e.g., in a bundle) to form the sensor arrangement 101. Such a grouping can make the sensor arrangement 101 more robust or less prone to failure. For example, grouping increases the collective cross-sectional area of the bases 104 making them less susceptible to stresses imparted on the sensor arrangement 101 (e.g., when the user's weight is placed on the sensor arrangement 101, when the garment 102 is stretched, etc.). Additionally, grouping can allow bases 104 to be collectively manipulated with ease during assembly, thereby making assembly of the user monitoring system 100 (e.g., positioning of the sensor arrangement 101 on the garment 102) easier and faster than assembly would be with individual bases 104.

In some variations, the sensor arrangement 101 may include a curved portion (e.g., the sensor arrangement 101 can have a curved configuration). For example, the conductive members 110 coupled to the bases 104 may be grouped together to form a curved path. In some variations, the curved portion of the sensor arrangement 101 can include a “S-shaped” configuration. In some variations, the curved portion of the sensor arrangement 101 can include a serpentine configuration. In some variations, the curved portion of the sensor arrangement 101 can include a coiled configuration.

A curved configuration can provide the benefit of reducing the strain on the sensor arrangement 101. For example, the curved configuration of the sensor arrangement 101 can provide excess length of conductive member 110 such that as the garment 102 is stretched (e.g., as the user is putting the garment 102 on or taking it off), the curved portion is straightened rather than the sensor arrangement 101 being placed under strain. Thus, the curved portion of the sensor arrangement 101 introduces a form of slack such that the garment 102 can be stretched without harming the sensor arrangement 101.

In locating the bases 104 (that include the respective sensors 108) to their respective measurement locations, the conductive members 110 coupled to the bases 104 can be bundled in a manner such that one or more bases 104 may diverge from the bundle. For example, in FIG. 9 (explained in detail below) base 904 substantially branches out from the bundle (e.g., bundle of conductive members coupled to the bases) in a manner such that base 904 is independently positioned at a desired measurement location (e.g., location at which sensor data is collected). In some variations, some of the bases 104 may diverge from the bundle while other bases 104 do not substantially diverge from the bundle. In some variations, none of the bases 104 may diverge from the bundle. In other variations, all of the bases 104 may diverge from the bundle in a manner that each base 104 navigates independently to a desired measurement location to collect sensor data. In one variation, a conductive member 110 coupled to a base 104 that diverges from the bundle may be longer in length than a conductive member 110 coupled to a base 104 that does not diverge from the bundle. It should be readily understood that many suitable configurations of the sensor arrangement 101 may be possible. For example, none, some, or all bases 104 (in any combination) may navigate to the desired respective measurement locations in series (in any permutation or order), in a non-diverging manner, to position the bases 104 in the sensor arrangement 101.

In some variations, the conductive members 110 coupled to the bases 104 may be arranged so as to distribute the bases 104 among a variety of discrete target regions of the garment, so as to collect sensor data (e.g., measure skin temperature, foot pressure, etc.) at different locations of interest when the garment is placed on a suitable part of the body of the user (e.g., foot of the user). In some variations, the measurement locations of interest can include locations typically experiencing high pressure when the user is active (e.g., walking or standing). For example, the sensor arrangement 101 may be constructed so as to arrange the one or more bases 104 on a sole region of the garment 102 (e.g., a sole region of a sock).

For example, the sensor arrangement 101 can be constructed so as to arrange one or more bases 104 on the sole region of the garment 102. In one exemplary variation, a sensor arrangement 101 can include six bases 104. A first base of the six bases may be positioned in a first region of the garment 102 configured to contact an Ossa digit of the foot, or toe. Three other bases of the six bases may be positioned in a second region of the garment 102 configured to contact the boundary of the phalanges and the metatarsals of the foot. Additionally, another base may be positioned in a third region of the garment 102 configured to contact the boundary of the metatarsals and the tarsals of the foot. Furthermore, another base may be positioned in a fourth region of the garment 102 configured to contact the heel of the foot. The configuration of bases 104 may be biased for a left foot in instances in which the garment 102 is configured to be placed on a left foot, while the configuration of bases 104 may be biased for a right foot in instances in which the garment 102 is configured to be placed on a right foot.

In other variations, the sensor arrangement 101 may be constructed so as to additionally or alternatively arrange one or more bases 104 on other regions of the garment (e.g., medial or lateral sides of a foot region of the garment, dorsal portion of a foot region of the garment, ankle region of the ankle, etc.).

In some variations, the sensor arrangement 101 may be similar to the arrangement of sensor leads and sensors described in U.S. patent application Ser. No. 16/836,800, the content of which is hereby incorporated by reference in its entirety.

In some variations, the sensor arrangement 101 can be enclosed in a protective material. For example, the conductive members 110 coupled to the bases 104 can be bundled and enclosed within a protective film (e.g., a plastic, such as a polyurethane film). The protective film can provide the benefit of protecting the sensor arrangement 101 from damage and/or outside moisture, and may be waterproof or water-resistant.

In some variations, the relative positions and orientations of some or all the bases 104 (and accordingly, the relative positions of the respective sensors 108) may be fixed with additional components. For example, once arranged in a configuration, at least some of the bases 104 and/or the conductive members 110 may be coupled to each other with epoxy or fasteners (e.g., ties, clips, etc.). Additionally or alternatively, at least some of the conductive members 110 coupled to the bases 104 may be collectively sealed together, such as sealed between multiple (e.g., upper and lower) layers of film or vacuum-sealed in a bag. Suitable film may be, for example, layers of polyurethane or other polymer. Accordingly, such sealing of the sensor arrangement 101 can set the arrangement of bases 104 during assembly.

In some variations, the sensor arrangement 101 can be coupled to the garment 102. For example, the garment 102 may have one or more recessed portions such that the sensor arrangement 101 can be position within the recessed portions. In some variations, the sensor arrangement 101 can be secured to the garment 102 with epoxy or other adhesive, sutures, connectors or fasteners, etc.

### Base

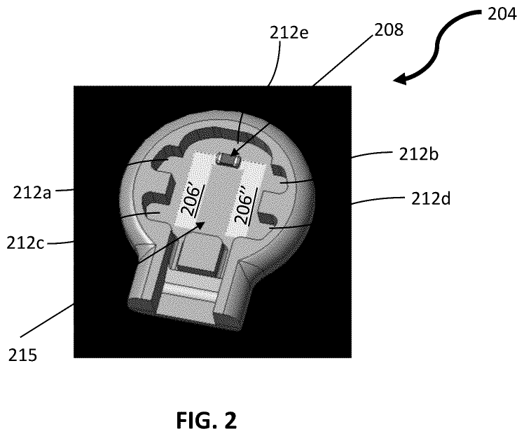

FIG. 2 is an exemplary variation of a base 204 (e.g., structurally and/or functionally similar to the base 104 in FIG. 1). The base 204 can include a recessed portion 215. In some variations, at least one conductive region, such as conductive region 106 in FIG. 1, can be formed within the recessed portion 215 of the base 204. For example, in FIG. 2, a first conductive region 206′ and a second conductive region 206″ can be formed within the recessed portion 215 of the base 204.

Additionally, the recessed portion 215 may include one or more cavities 212 (e.g., cavities 212a-212e, collectively cavity 212). At least one sensor 208 (e.g., structurally and/or functionally similar to sensor 108 in FIG. 1) can be coupled to the base 204 via the at least one conductive region. For example, in FIG. 2, at least one sensor 208 can be coupled to one or both of the first conductive region 206′ and the second conductive region 206″. Although, FIG. 2 shows a single sensor 208 coupled to both the first conductive region 206′ and the second conductive region 206″, in some variations, one sensor may be coupled to the first conductive region 206′ while another sensor may be coupled to the second conductive region 206″.

The base 204 can be manufactured using a suitable manufacturing process (e.g., casting, molding, machining (e.g., milling), 3D printed, etc.). For example, the base 204 may be manufactured using insert molding and hot stamping. In some variations, the base 204 can be manufactured using Laser Direct Structuring (LDS). In some variations, the base 204 can be a thermoplastic part that is integrated with electronic circuit traces. The base 204 may comprise any suitable material, such as a rigid or semi-rigid plastic (e.g., nylon or acrylonitrile-butadiene-styrene (ABS)).

In some variations, the base 204 may be manufactured individually. Put differently, each base 204 in a sensor arrangement (e.g., sensor arrangement 101 in FIG. 1) may be manufactured individually before being grouped together to form the sensor arrangement. For example, each base 204 in a sensor arrangement may be manufactured individually using an LDS process and then grouped together. Alternatively, in some variations, the base 204 may be formed as a part of an interconnected array of bases. For example, an interconnected array of bases may be manufactured using a molding process, such as the LDS process. The base 204 can then be disjoined (e.g., split, removed, etc.) from the interconnected array of bases following the manufacturing process. Each of the disjointed bases (e.g., base 204) may be grouped together (e.g., via one or more conductive members) to form a sensor arrangement (e.g., sensor arrangement 101 in FIG. 1).

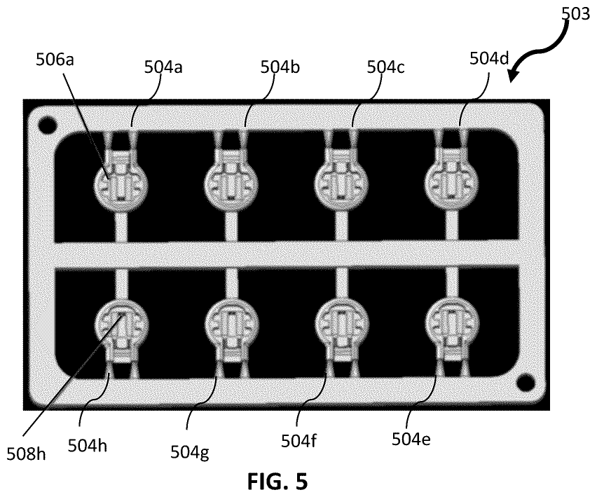

In some variations, the base 204 can be disjoined after a respective conductive region (e.g., the first conductive region 206′ and/or the second conductive region 206″ in FIG. 2) has been formed in each base of the interconnected array of bases. In some variations, the base 204 can be disjoined after a sensor 208 has been coupled to the respective conductive region within each base of the interconnected array of bases. An exemplary variation of an interconnected array of bases is illustrated in FIG. 5 discussed in detail below.

In some variations, the recessed portion 215 may be formed within the base 204 during the manufacturing process of the base 204. For example, the recessed portion 215 may be formed during the manufacturing of an interconnected array of bases before the base 204 is disjoined from the interconnected array of bases. In some variations, the recessed portion 215 may be formed after the base 204 has be manufactured. For example, the recessed portion 215 may be formed by machining the base 204 after the base 204 has been disjoined from an interconnected array of bases.

The shape of the recessed portion 215 can be configured to increase contact between an encapsulating material (e.g., gluing material such as ultraviolet resin, epoxy resin, etc.) and the base 204. For example, the shape of recessed portion 215 can include cavities 212 to increase surface area of contact between the encapsulating material and the base 204. This can increase the strength of the base 204, provide flexibility, and make the base 204 moisture resistant. For example, the encapsulating material may be a hardener that can increase the mechanical strength of the base 204 giving the base 204 good fatigue resistance. Furthermore, the encapsulating material can improve the coupling between the sensor 208, the conductive member 210, and the base 204. Although tough, the encapsulating material can be flexible in nature. The encapsulating material can provide flexibility to the coupling between the sensor, the conductive member 210, and the base 204 making it less at risk of breakage. Additionally, the encapsulating material can act as a sealant by sealing any voids in the base 204 making it moisture resistant.

In one variation, the shape of the recessed portion 215 can include cavities 212 in a manner such that the cavities 212 have bilateral symmetry to help maintain, for example, uniformity of adhesion between the encapsulating material and the base 204. In another variation, the shape of the recessed portion 215 can include cavities in a manner such that the cavities have radial symmetry to help maintain, for example, uniformity of adhesion between the encapsulating material and the base 204.

In one variation, the shape of the recessed portion 215 can include a central cavity 212e adjoining at least one peripheral cavity (e.g., peripheral cavity 212a and/or peripheral cavity 212b). The recessed portion 215 can be formed in such a manner that the peripheral cavity 212a and peripheral cavity 212b are opposite each other and across the central cavity 212e. In some variations, the peripheral cavities 212a and 212b may include “C-Shaped” cavities. The C-shape of the peripheral cavities 212a and 212b may face each other.

In addition, the shape of the recessed portion 215 can include peripheral cavity 212c and peripheral cavity 212d. The peripheral cavity 212c and peripheral cavity 212d can be opposite each other and across the central cavity 212e. For instance, peripheral cavity 212a and peripheral cavity 212c can be on a first side of the central cavity 212e while peripheral cavity 212b and peripheral cavity 212d can be on a second side (e.g., opposite to the first side) of the central cavity 212e. The recessed portion 215 can be formed in such a manner that peripheral cavity 212a and peripheral cavity 212c are be spaced apart (i.e., they are not adjoining) from each other. In a similar fashion, peripheral cavity 212b and peripheral cavity 212d can be spaced apart from each other. In some variations, the peripheral cavities 212c and 212d may include “C-Shaped” cavities. The C-shapes of the peripheral cavities 212c and 212d may face each other.

At least one conductive region, such as a first conductive region 206′ and a second conductive region 206″—can be formed in the recessed portion 215. The first conductive region 206′ and/or the second conductive region 206″ can be formed by applying (e.g., spraying, brushing, etc.) a conductive material (e.g., tin, copper, nickel, etc.) in the recessed portion 215. For example, the first conductive region 206′ and/or the second conductive region 206″ can be formed by applying tin in the recessed portion 215. The application of tin to form the first conductive region 206′ and/or the second conductive region 206″ can be through a spraying or brushing process, for example.

In some variations, the first conductive region 206′ and the second conductive region 206″ may be formed in a manner such that a non-conductive region can exist in between the first conductive region 206′ and the second conductive region 206″. In some variations, the first conductive region 206′ and the second conductive region 206″ can be “L-shaped” paths. One end of the first conductive region 206′ (i.e., the end of the L-shaped path 206′) can be adjoining one end of the second conductive region 206″ (i.e., the end of the L-shaped path 206″).

In one variation, a first end of the sensor 208 is coupled to the first conductive region 206′ and a second end of the sensor 208 is coupled to the second conductive region 206″. Current can flow into the sensor 208 along one conductive region and current can flow out of the sensor 208 along the other conductive region. For instance, current can flow into the sensor 208 along the first conductive region 206′ and current can flow out of the sensor 208 along the second conductive region 206″. Alternatively, current can flow into the sensor 208 along the second conductive region 206″ and current can flow out of the sensor 208 along the first conductive region 206′. In some variations, the sensor 208 can be soldered into the conductive region 206 such that a first end of the sensor is soldered to the first conductive region 206′ and a second end of the sensor is soldered to the second conductive region 206″.

The sensor 208 may be any suitable type of sensor, such as a temperature sensor, pressure sensor, a moisture sensor, a strain sensor, a weight sensor, a movement/activity sensor, a muscle activity (e.g., EMG) sensor, an orientation sensor, a sensor that tracks the amount of time a garment is worn (e.g., to determine compliance with a treatment plan), etc. In some variations, the sensor 208 can be a temperature sensor, such as thermistors, thermocouples, or other suitable temperature sensors. In some variations, the sensor 208 can be a Negative Temperature Coefficient (NTC) thermistor.

The base 204 can be designed by factoring in the comfort of the user. For example, in some variations, the base 204 can be at least partially circular in shape so as to reduce the contact area when the base 204 is coupled to the user. Since the contact area is reduced, the user may not feel the base 204 while wearing a garment that is coupled to the base 204. In some variations, the side of the base 204 comprising the encapsulating material can be configured to be closer to the user's skin. Put differently, the base 204 can be coupled to the garment in such a manner that the encapsulating material can be configured to be closer to the user's skin when the garment is worn by the user.

### Conductive Member

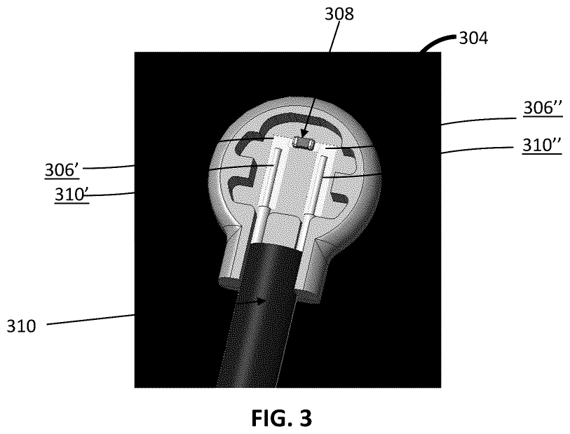

FIG. 3 is an exemplary variation of a base 304 illustrating elements of a conductive member 310 (e.g., structurally and/or functionally similar to conductive member 110 in FIG. 1) coupled to the base 304 (e.g., structurally and/or functionally similar to base 204 in FIG. 1). As seen in FIG. 3, elements of the conductive member 310, such as conductive member element 310′ and conductive member element 310″ can be coupled to at least one conductive region, such as a first conductive region 306′ and second conductive region 306″ formed within the base 304.

As described in FIG. 2, the at least one conductive region can include a first conductive region 306′ (structurally and/or functionally similar to conductive region 206′ in FIG. 2) and a second conductive region 306″ (structurally and/or functionally similar to conductive region 206″ in FIG. 2). A first conductive member element 310′ of the conductive member 310 can be coupled to the first conductive region 306′ while a second conductive member element 310″ of the conductive member 310 can be coupled to the second conductive region 306″.

The conductive member 310 can be coupled to the sensor 308 (e.g., structurally and/or functionally similar to sensor 208 in FIG. 2) of the base 304 via the first conductive region 306′ and/or the second conductive region 306″. Put differently, current can flow from the first conductive member element 310′ of the conductive member 310 via the first conductive region 306′ into the sensor 308. Current can flow out from the sensor 308 via the second conductive region 306″ into the second conductive member element 310″ of the conductive member 310. Alternatively, current can flow from the second conductive member element 310″ of the conductive member 310 via the second conductive region 306″ into the sensor 308. Current can flow out from the sensor 308 via the first conductive region 306′ into the first conductive member element 310′ of the conductive member 310.

In some variations, each of the first conductive member element 310′ and the second conductive member element 310″ can be coupled to the first conductive region 306′ and/or the second conductive region 306″ using any suitable coupling method (e.g., using alligator clips, electrical tape, hot glue, crimping, soldering, etc.). For example, the first conductive member element 310′ of the conductive member 310 can be soldered into the first conductive region 306′. In a similar manner, second conductive member element 310″ of the conductive member 310 can be soldered in the second conductive region 306″.

In some variations, the conductive member 310 can include braided wires made of conductive material that is encased in an insulating cover. Each wire of the braided wires can be a conductive element of the conductive member 310. For example, in FIG. 3, the conductive member 310 can include the first conductive member element 310′ and second conductive element 310″ braided together and encased in an insulating cover (e.g., sheath). For instance, the conductive member 310 can include braided electrical wires (e.g., copper wire, aluminum wire, etc.) encased in a thermoplastic insulation (e.g., PVC (polyvinyl chloride) insulation, PE (polyethylene), etc.).

In one variation, the conductive member 310 can be cut to a required length (e.g., using a wire trimming process). The insulating cover of the conductive member 310 can be removed (e.g., using wire stripping process) from a portion of one end of the conductive member. The exposed portion of the conductive member 310 can be unbraided. In some variations, when unbraided, the exposed portion of conductive member 310 includes portions of the first conductive member element 310′ and the second conductive member element 310″ as separate individual elements. Put differently, the non-exposed portion of the conductive member 310 may include portions the first conductive member element 310′ and the second conductive member element 310″ braided together, while the exposed portion of the conductive member 310 may include portions of the first conductive member element 310′ and the second conductive member element 310″ as separate unbraided portions. These separate unbraided portions of the first conductive member element 310′ and the second conductive member element 310″ can be tinned to protect them from corrosion or oxidation. The tinned part of the first conductive member element 310′ can be coupled to the first conductive region 306′ and the tinned part of the second conductive member element 310″ can be coupled to the second conductive region 306″. For example, the tinned part of the first conductive member element 310′ can be soldered into the first conductive region 306′ while the tinned part of the second conductive element can be soldered into the second conductive region 306″. Although in this particular variation, the sequence of operations described is trimming of the conductive member 310, removal of the insulating cover, and unbraiding the exposed portion of the conductive member 310, it should be readily apparent that the sequence of operations may be performed in any order. For instance, in another variation, the insulating cover may be removed from one end of a conductive member 310 before trimming the conductive member 310.

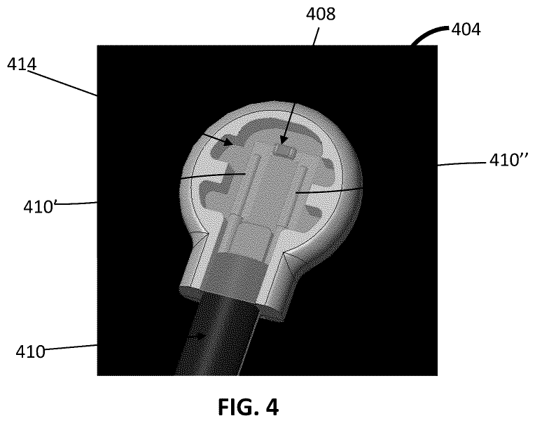

Although, FIGS. 3-4 show a variation in which the conductive member elements are grouped (e.g., braided) together as a conductive member 310, it should be understood that alternatively, one or more conductive member elements may be separate individual conductive members. For example, in one variation, the first conductive member element 310′ can be a separate conductive member itself. Put differently, the first conductive member element 310′ need not be braided together with the second conductive member element 310″ and then encased in an insulating material. Instead, the first conductive member element 310′ can be separately encased in an insulating material. In a similar manner, the second conductive member element 310″ can be separately encased in an insulating material, thereby making the first conductive member element 310′ a first conductive member and the second conductive member element 310″ a second conductive member.

### Encapsulating Material

FIG. 4 is an exemplary variation of a base 404 illustrating an encapsulating material 414 encapsulating a sensor 408 (e.g., structurally and/or functionally similar to sensor 108 in FIG. 1) and a conductive member 410 (e.g., structurally and/or functionally similar to conductive member 110 in FIG. 1) within the base 404 (e.g., structurally and/or functionally similar to base 104 in FIG. 1).

In some variations, after the conductive member 410 and the sensor 408 have been coupled to the conductive region (not shown in FIG. 4), an encapsulating material 414 (e.g., adhesives such as ultraviolet resin, epoxy resin, polyurethane, polyimides, etc.) can be applied to the recessed portion (e.g., recessed portion 215 in FIG. 2) of the base 404. For example, the encapsulating material 414 can be dispensed, spread, or applied to the recessed portion of the base 404. In some variations, the base 404 can be subjected to heat (e.g., via a tunnel stove) to further strengthen the encapsulating material 414.

The encapsulating material 414 can protect the coupling (e.g., the soldering) between the sensor 408 and the conductive region and the coupling between the conductive member 410 and the conductive region. In addition, the encapsulating material 414 can strengthen these coupling. The adhesive properties of the encapsulating material 414 can help seal any gaps or voids in the base 404, thereby making the base 404 moisture-resistant. The encapsulating material 414 can provide flexibility, eliminate corrosion, and provide good fatigue resistance to the base 404. Additionally, in some variations, the encapsulating material 414 can be electrically and/or thermally insulating, thereby protecting the user.

### Making a User Monitoring System

In one variation, a method of making a user monitoring system (e.g., user monitoring system 100 in FIG. 1) can include making a sensor arrangement (e.g., sensor arrangement 101 in FIG. 1), preparing a garment to receive the sensor arrangement (e.g., garment 102 in FIG. 1), and positioning at least a part of the sensor arrangement in or on the garment and/or coupling the sensor arrangement to the garment.

### Making a Sensor Arrangement

As discussed above, the sensor arrangement can include multiple conductive members that are coupled to their respective bases all of which are grouped together. Put differently, each base in the sensor arrangement may be coupled to a respective conductive member. One or more of such coupled bases and conductive members can be grouped (e.g., bundled) together to form the sensor arrangement.

In one variation, a sensor module may comprise a single base coupled to a respective conductive member. For example, in FIG. 1, base 104a coupled to conductive member 110a may be a first sensor module. In a similar manner, base 104n coupled to conductive member 110n may be a second sensor module. Accordingly, grouping the sensor modules together can form the sensor arrangement.

In one variation, the sensor arrangement may comprise a single sensor module. In other variations, the sensor arrangement may comprise two or more sensor modules.

### Making a Base

In one variation, a method of making a base (e.g., base 104 in FIG. 1) can include manufacturing the base individually using a suitable manufacturing process (e.g., casting, molding, etc.). For example, the base may be manufactured using Laser Direct Structuring (LDS). For instance, the base can include a thermoplastic part that is integrated with electronic circuit traces using LDS. In some variations, the base can include two or more separate parts that are fused together. For example, the base can include a first part that comprises electronic circuit traces and a second part that comprises plastic providing a mechanical function to the base. The first part and the second part can be fused together (e.g., using two-shot molding process) to form the base. Each base can be individually manufactured and then coupled to their respective conductive members to form corresponding sensor modules. The sensor modules can then be grouped together to form the sensor arrangement.

Alternatively, multiple bases can be manufactured together as an interconnected array of bases. For example, an interconnected array of bases can be manufactured using a molding process, such as LDS in a manner such that each base in the interconnected array of bases is similar to each other base in the interconnected array of bases. FIG. 5 is an exemplary variation of an interconnected array 503 of bases. As seen in FIG. 5, the interconnected array 503 can comprise multiple similar (e.g., similar in structure and/or similar in function) bases 504a-504h that are connected together. The interconnected array 503 of bases in FIG. 5 is a panel of bases. Put differently, the interconnected array 503 includes a peripheral frame with one of more spines connected to the peripheral frame. During the manufacturing of the interconnected array 503 of bases, each base is formed on a spine of the interconnected array 503 of bases. More than one base can be formed on a single spine. For instance, in FIG. 5, two bases are formed on each spine of the interconnected array 503 of bases.

Although, FIG. 5 illustrates the interconnected array 503 as a panel, it should be readily apparent that the interconnected array 503 can be manufactured to be of any suitable array layout (e.g., rectangular array, hexagonal array, radial array, etc.).

Conductive material may be applied to each base 504a-504h of the interconnected array 503 to form respective conductive regions in each base. For example, in FIG. 5, application of conductive material to each base in the interconnected array 503 forms the conductive region 506a in base 504a. The application of conductive material can comprise applying (e.g., spraying, brushing, etc.) tin or other suitable conductive material to each base in the interconnected array 503. Additionally, a sensor may be coupled to each respective conductive region of each base 504a-504h in the interconnected array 503. For example, in FIG. 5, coupling sensors to each base in the interconnected array 503 results in sensor 508h being coupled to the base 504h. Coupling a sensor to each base in the interconnected array 503 can comprise soldering the sensor to the respective conductive region in the base.

Although the interconnected array 503 in FIG. 5 comprises eight bases 504a-504h, it should be readily apparent that the interconnected array 503 can be manufactured to comprise any number of bases (e.g., at least two bases, at least three bases, at least four bases, at least five bases, etc.). As discussed above, in one variation, the interconnected array 503 of bases can be a panel of bases.

Although the same type of sensor may be coupled to each base 504a-504h in the interconnected array 503 of bases, in some variations, the interconnected array 503 of bases can comprise more than one type of sensor. Put differently, some bases in the interconnected array 503 of bases may be coupled to one type of sensor while other bases in the interconnected array 503 of bases may be coupled to another type of sensor. For example, in FIG. 5, bases 504a-504d may be coupled to a temperature sensor while bases 504e-504h may be coupled to a pressure sensor.

After making an interconnected array 503 of bases, each base can be disjointed from the interconnected array 503 to form individual bases. For example, each base can be split from the interconnected array 503 to form individual bases. Making individual bases in this manner may improve the consistency and reliability of the sensor arrangement. Put differently, each base in the sensor arrangement may be constructed substantially the same as each other base in the sensor arrangement (or portion of the sensor arrangement), as the bases were manufactured together as an interconnected array under similar conditions. Additionally, manufacturing an interconnected array of bases can make the method of making a base scalable for manufacturing larger quantities of bases efficiently and reliably. For example, any number of bases can be manufactured together in a consistent and reliable manner by making a single interconnected array 503 of bases.

After splitting the individual bases from the interconnected array of bases, the individual bases can be coupled to a corresponding conductive member to form a corresponding sensor module. These sensor modules can be grouped together to form the sensor arrangement.

### Making a Sensor Module

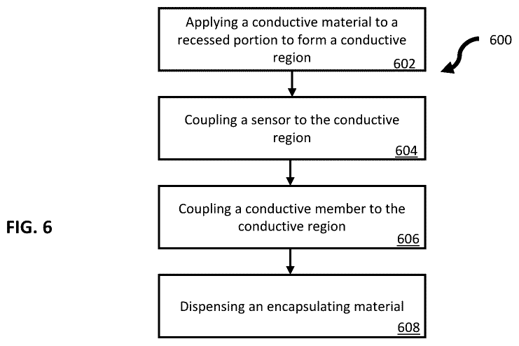

FIG. 6 is a flowchart of an exemplary variation of a method 600 of making a sensor module (e.g., base 104a coupled to conductive member 110a in FIG. 1 or base 104n coupled to conductive member in 110n in FIG. 1).

At 602, a conductive material can be applied to a recessed portion (e.g., recessed portion 215 in FIG. 2) of a base (e.g., base 204 in FIG. 2) to form at least one conductive region (e.g., conductive region 206 in FIG. 2). In one variation, the conductive material may be applied when the conductive material is being applied to an interconnected array of bases (e.g., interconnected array 503 in FIG. 5). In another variation, individual bases may be disjoined from an interconnected array of bases (e.g., interconnected array 503 in FIG. 5) and then the conductive material may be applied to each individual base. The at least one conductive region can include two conductive regions that are adjoining. In one variation, a non-conductive region may exist between the two adjoining conductive regions.

At 604, a sensor can be coupled to the at least one conductive region. In one variation, the sensor can be soldered into the at least one conductive region. The sensor may be coupled in such a manner that one end of the sensor is coupled to a first conductive region in the at least one conductive region while another end of the sensor is coupled to a second conductive region in the at least one conductive region. In one variation, the sensor may be coupled when sensors are being coupled to each base in an interconnected array of bases (e.g., interconnected array 503 in FIG. 5). In another variation, individual bases may be disjoined from an interconnected array of bases (e.g., interconnected array 503 in FIG. 5) after the application of the conductive material. The sensor may then be coupled to the respective conductive regions of each individual bases.

In some variations, after step 604, individual bases from an interconnected array of bases (e.g., interconnected array 503) are disjointed. At 606, a conductive member (e.g., conductive member 310 in FIG. 3) can be coupled to the at least one conductive region of the base. In one variation, a portion of the conductive member can be soldered into the conductive region of the base. At 608, an encapsulating material (e.g., ultraviolet resin, epoxy resin, etc.) can be applied to the recessed portion to encapsulate the sensor and the conductive member within the base.

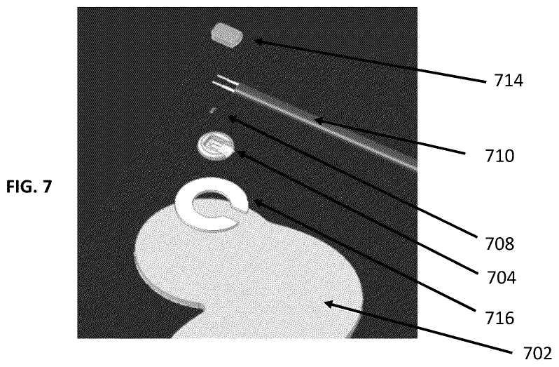

FIG. 7 is an exemplary variation of an exploded view of a sensor module being coupled to a garment 702. As shown in FIG. 7, a base 704 can include a recessed portion. A conductive region can be formed in the recessed portion by applying conductive material to the recessed portion. A sensor 708 can be coupled to the conductive region within the recessed portion. A conductive member 710 can be coupled to the conductive region of the base. An encapsulating material 714 can be applied to encapsulate the sensor 708 and the conductive member 710 within the base 704. In one variation, the sensor module may be sealed to the garment 702 using a sealant 716 (e.g., silicone). The sealant 716 can have adhesive properties which can attach to the garment, thereby coupling the sensor module to the garment 702. In some variations, the sealant 716 can be configured to withstand high and low temperatures. In some variations, the sealant 716 can be configured to be resistant to chemicals and other moistures.

### Making Sensor Arrangement(s)

As illustrated in FIG. 7, in some variations, a single sensor module can form a sensor arrangement that is coupled to the garment. Alternatively, sensor modules may be grouped (e.g., bundled) together to form the sensor arrangement. The bundle may be shaped in any suitable manner such that each base within the sensor arrangement can contact various locations of interest on a suitable part of the body of the user. For example, the bundle may be shaped in a manner such that each base within the sensor arrangement contacts pressure points on a foot of a user so as to collect sensor data from these points.

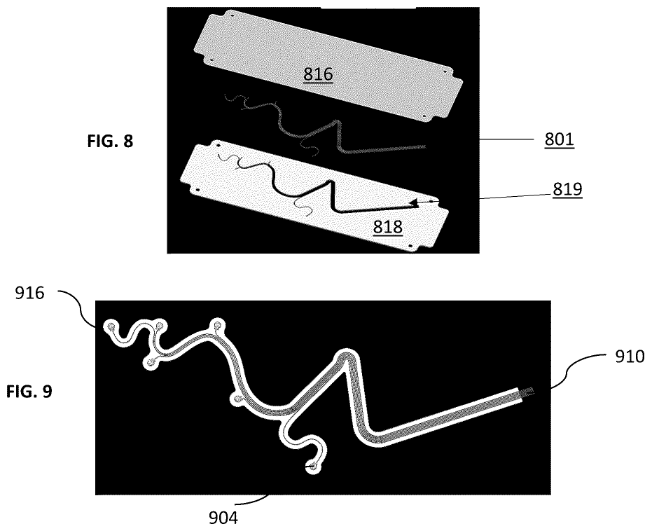

There can be several suitable methods for arranging the sensor modules into a bundled sensor arrangement. FIG. 8 shows an exemplary variation of a method of arranging the sensor modules into a bundle. In one variation, the sensor modules 801 may be arranged (e.g., manually or automatically with tooling, etc.) with the assistance of a fixture 818 having one or more open channels (e.g., groove 819) that is shaped to resemble the desired sensor arrangement layout. Sensor modules may be placed into the open channels (e.g., groove 819), which gathers the sensor modules and shapes the sensor arrangement into the desired shape. Similarly, in another exemplary variation, a fixture (not shown in FIG. 8) may include one or more closed channels (e.g., lumens) shaped to resemble the desired sensor arrangement layout. In this variation, sensor modules may be fed longitudinally into the closed channels, which similarly gathers and shapes the sensor modules into the desired shape. In yet another exemplary variation, the modules may be manually arranged in a free-form manner on a surface. The surface may include, for example, an outline or reference markers guiding placement of the modules.

In some variations, after the sensor modules are arranged into a bundle or other suitable pattern, the sensor arrangement can be sealed or otherwise set, in order to fix the relative positions of the bases. In one variation, the bundle is first placed in the slot 819 of the fixture 818. A protective film 816 is then attached onto the fixture 818, thereby covering one portion, such as a top portion or a bottom portion of the sensor arrangement. The protective film 816 may fix the sensor arrangement in a manner such that relative positions of the bases in the sensor arrangement are fixed. The protective film 816 may include adhesive properties so as to attach to the fixture 818. Alternatively, certain portions of the protective film 816 may include an adhesive to attach the protective film 816 to the fixture 818. Once the relative positions of the bases in the sensor arrangement are fixed and a top or a bottom portion of the sensor arrangement is covered with the protective film, the fixture 818 can be detached (e.g., removed) from the protective film 816. The portion of the sensor arrangement not covered with the protective film 816 is further covered with more protective film. For instance, if a first portion of the protective film 816 covers a top or a bottom portion of the sensor arrangement, then a second portion of the protective film can be used to cover the portion of the sensor arrangement that is not already covered.

In one variation, any portions of the protective film not covering the sensor arrangement can be removed (e.g., using a die cut process). Alternatively, after the protective film covers a top or a bottom portion of the sensor arrangement and prior to sandwiching the sensor arrangement with more protective film (e.g., attaching protective film to a portion of the sensor arrangement that is not already covered), the protective film is cut using a die cut process. For example, the protective film can be cut in a shape of the sensor arrangement so as to seal the sensor arrangement without having any extra portion of protective film not covering the sensor arrangement. The resultant sensor arrangement sealed in the protective film can be seen in FIG. 9. As seen in FIG. 9, the sensor arrangement can be sealed in a protective film 916 (e.g., polyurethane film).

In other variations, after the sensor modules are arranged into a bundle, the sensor arrangement can be laminated between layers of thermoplastic film (e.g., thermoplastic polyurethane, or other thermoplastic), such as with heat sealing. Other examples of fixing the sensor arrangement include sealing in a bag (e.g., vacuum sealing), and coupling sensor modules together with adhesive, fasteners, threads, etc.

As discussed above and seen in FIG. 9, the sensor arrangement can include a curved configuration to reduce stress. In some variations, the sensor arrangement can be an S-shaped arrangement. In some variations, the sensor arrangement can have a serpentine configuration.

### Making a Garment

In some variations, the method of making a garment may be similar to the method described in U.S. patent application Ser. No. 16/836,800, the content of which is hereby incorporated by reference in its entirety.

In one variation, a garment (e.g., garment 102 in FIG. 1) may be prepared by forming one or more recessed portions, such that the recessed portions may be configured to receive the sensor arrangement. For example, the garment may include a plush material (e.g., terry knit) that can be thinned or cut shorter (e.g., with a cutting blade, scissors, a razor, and the like) in selected regions where a recessed portion is desired. In some variations, the one or more recessed portions of the garment may be arranged on an internal surface of the garment. In other variations, the one or more recessed portions may be arranged on an external surface of the garment. In one variation, the recessed portions may resemble a shape of the sensor arrangement. In one variation, the recessed portions may have any suitable size and shape for receiving at least a part of the sensor arrangement.

### Forming a Cover

As discussed above, in one variation, the user monitoring system described herein can include a cover. In some variations, the cover may include a flexible material that may formed into a suitable shape for covering the sensor arrangement and coupling to the garment. The cover may include a single portion or multiple portions. In an exemplary variation, the cover may include a material conducive to heat sealing, such as a textile (e.g., polyester or other suitable fabric) lined with a thermoplastic (e.g., thermoplastic polyurethane). However, in other variations the cover may include a textile otherwise coupleable to the garment via epoxy, fasteners, etc. Generally, a textile cover may be formed by cutting the cover shape from the textile (e.g., with scissors, laser cutting, etc.). Accurate cutting of the cover shape may be facilitated with a template, jig, and/or other suitable tooling. Suitable exemplary cover shapes are described in further detail above.

### Example Method of Making a Sensor Arrangement

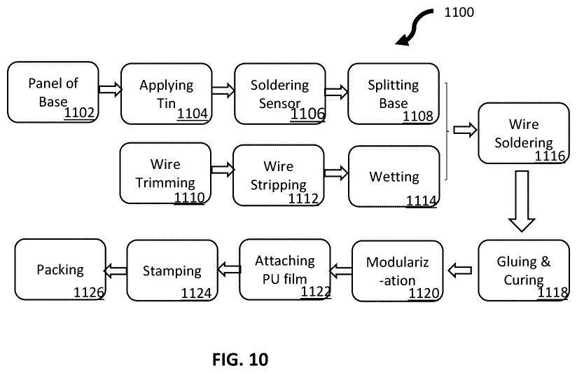

FIG. 10 is an exemplary variation of a method 1100 of making a sensor arrangement (e.g., sensor arrangement 101 in FIG. 1) for a user monitoring system (e.g., user monitoring system 100 in FIG. 1).

At 1102, the method includes making an interconnected array (e.g., interconnected array 503 in FIG. 5) of bases (e.g., base 104 in FIG. 1). In one variation, the interconnected array of bases can be manufactured using LDS. For example, a thermoplastic material that is doped with a non-conductive metallic inorganic compound may be used to manufacture the interconnected array of bases. The thermoplastic material with the non-conductive inorganic compound is molded using injection molding. Then, the molded part is activated using laser activation. The laser activation breaks the non-conductive atoms away from the conductive atoms, exposing them and selectively activating the material for metallization. At this point, electronic circuit traces are etched into the material. Finally, the electronic circuit traces are metallized using electroless plating. In some variations, the interconnected array can be a panel as seen in FIG. 5. The interconnected array of bases can have any number of bases that are manufactured together.

At 1104, a conductive material can be applied to each base in the interconnected array (e.g., panel) of bases to form a respective conductive region (e.g., conductive region 106 in FIG. 1) in each base. For example, conductive material can be applied to each base in the interconnected array of bases in a serial manner until all the bases have at least one conductive region. Alternatively, conductive material can be applied to one or more bases in the interconnected array of bases in a parallel manner until all the bases have at least one conductive region. In some variations, conductive material is applied in an “L-shaped” manner in each base to form a first conductive region. Subsequently, additional conductive material is applied once again in “L-shaped” manner in each base to form a second conductive region. The second conductive region is formed such that it is adjoining the first conductive region and the end of the L-shape of the first conductive region meets the end of the L-shape of the second conductive region.

In some variations, tin can be sprayed onto a recessed portion in each base in the interconnected array of bases to form the respective conductive region. In some variations, tin can be brushed into the recessed portion in each base in the interconnected array of bases to form the respective conductive region. Tin can be applied in a manner such that the conductive region can include a first conductive region and a second conductive region adjoining each other.

At 1106, a sensor (e.g., sensor 108 in FIG. 1) can be coupled to the respective conductive region of each base in the interconnected array of bases. For example, a sensor can be soldered into the respective conductive region. The sensor can be soldered in a manner such that one end of the sensor is soldered into the first conductive region and the other end of the sensor is soldered into the second conductive region of the base. In some variations, the sensor may be a NTC thermistor.

Once the sensors are soldered into their respective conductive region of each base, the quality of the soldering can be checked. For example, a visual inspection (e.g., using a magnifying glass, wiggling the interconnected array of bases, etc.) may be conducted to check the quality of the soldering. If at least one sensor seems loose, some or all of the sensors may be re-soldered into their respective conductive regions. In one variation, an automated visual inspection may be conducted to check the quality of soldering. For example, a camera may autonomously scan the interconnected array of bases for defects and failure. In another variation, a multimeter may be used to check the quality of soldering (e.g., checking for resistance).

At 1108, each base from the interconnected array of bases can be split into individual bases. Although, in this example, individual bases are split from the interconnected array of bases after applying the conductive material and coupling the sensor to the base, it should be readily apparent that the individual bases may be split at any suitable point. For example, the individual bases may be split from the interconnected array of bases prior to applying the conductive material. Alternatively, the individual bases may be split from the interconnected array of bases prior to coupling the sensor but after the application of the conductive material.

At 1110, a conductive member (e.g., conductive member 110 in FIG. 1) can be cut to a required length. For example, the conductive member can include two or more wires (e.g., copper wire, aluminum wire, etc.) that are braided together. The braided wires may be covered with an insulating material. The conductive member (e.g., braided wires with the insulation) can be trimmed (i.e., cut) to a desired length (e.g., using a trimming process). The desired length of the conductive member may be determined based on the requisites for the sensor arrangement. For instance, the length of the conductive member may be determined by the amount of curvature that the sensor arrangement is supposed to have. In another example, the length of the conductive member may be determined based on the position of the base that it is coupled to in the sensor arrangement. For instance, if the base is supposed to be located in a distal position substantially diverging away from the bundle then the length of the conductive member may be substantially longer. However, if the base does not diverge away from the bundle then the length of the conductive member may be shorter.

At 1112, insulation material may be removed from one end of the conductive member (e.g., using wire stripping). For example, as discussed above, the conductive member can have two conductive member elements (e.g., two wires) that are braided, wound, or otherwise grouped together. When insulation is stripped from a portion on one end of the conductive member it exposes the two conductive member elements (in their braided form). So, a portion of the two conductive member elements are exposed. Now, this portion of the respective conductive member elements can be unbraided. Therefore, this portion of the respective conductive member elements become individual separate elements.

At 1114, tin can be added to the respective portions of the individual conductive member elements in order to protect the conductive member from corrosion. For instance, tin can be applied to the individual separate portions of the wires at the end of the braided wires (e.g., wetting process) to protect the individual separate portions of the wires from corrosion.

At 1116, the respective portions of the individual conductive member elements can be coupled to the conductive region of the base. For example, the individual separate portions of the wires at the end of the braided wire that were wetted can be soldered to a conductive region of a base. To do so, the base may be fixed on a carrier of a hot-bar soldering machine. The two individual separate portions of the wires may be fixed in the conductive region of the base (e.g., using a fixture). The soldering probe of the hot-bar soldering machine can then solder the individual separate portions of the wires to the conductive region of the fixture.

At 1118, an encapsulating material can be dispensed in the recessed portion of the base to encapsulate the sensor and the conductive member. For example, an adhesive and/or gluing material such as ultraviolet resin or epoxy resin can be dispensed in the recessed portion of the base. To further strengthen the gluing material, heat can be applied (e.g., using tunnel stove) to the gluing material. In this manner, the gluing material can protect the components (e.g., the soldered sensor and/or the soldered wires) in the base.

In one variation, the encapsulation of the sensor and the conductive member within the base can be checked by placing the base in a water tank. For example, after placing the base in the water tank, the functioning of the sensor may be tested. If the sensor functions as it is supposed to function, the base is water-proof and the quality of the encapsulation can be considered to be good. However, if the sensor does not function as it is supposed to function, then there may be an issue with the encapsulation. In such a situation, the base may have to be encapsulated again as described in step 1118.

At 1120, the sensor modules (e.g., bases coupled to their respective conductive members) can be routed through an opening (e.g., slot) in a fixture to form a sensor arrangement. The opening can be of a predetermined shape. For example, at least a portion of the opening can have a curved path. The sensor modules may be routed manually. Alternatively, the sensor modules may be routed automatically with tooling.

At 1124, a protective film (e.g., polyurethane film) can be attached to the top portion and the bottom portion of the sensor arrangement. In one variation, a first protective film can be attached to the fixture when the sensor modules are in the slot within the fixture. The first protective film may fix the sensor modules in a specific way such that the relative positions of the bases are fixed. The fixture can then be removed such that a top portion and/or a bottom portion of the sensor arrangement is covered with the first protective film. A second protective film can be attached to a portion that is opposite to the portion of the sensor arrangement that is covered with the first protective film. For example, if the first protective film covers a top portion of the sensor arrangement then the second protective film can be attached to a bottom portion that is opposite to the top portion. Alternatively, if the first protective film covers a bottom portion of the sensor arrangement then the second protective film can be attached to a top portion that is opposite to the bottom portion. The protective film at step 1114 is attached in such a manner that the entire sensor arrangement is sealed in the protective film. For example, the first protective film and the second protective film together seal the sensor arrangement. Put differently, the sensor arrangement is sandwiched between the first protective film and the second protective film. In this manner, the entire sensor arrangement can be sealed in the first protective film and the second protective film.

At 1124, portions of the first protective film and the second protective film that are not in contact with the sensor arrangement can be removed using a die cut process. At 1126, the sensor arrangement enclosed in the protective film can be packaged and coupled to a garment.

It should be readily apparent that the steps 1102-1124 of the method 1100 can be performed in any order. For instance, in one variation, step 1108 may be performed before step 1104. In another variation, step 1108 may be performed after step 1104 but before step 1106. In still another variation, step 1112 may be performed before step 1110. In another variation, steps 1110-1114 may be performed before 1102-1108. Put differently, the sequence of the steps described in method 1100 may be performed in any order.

The foregoing description, for purposes of explanation, used specific nomenclature to provide a thorough understanding of the invention. However, it will be apparent to one skilled in the art that specific details are not required in order to practice the invention. Thus, the foregoing descriptions of specific embodiments of the invention are presented for purposes of illustration and description. They are not intended to be exhaustive or to limit the invention to the precise forms disclosed; obviously, many modifications and variations are possible in view of the above teachings. The embodiments were chosen and described in order to explain the principles of the invention and its practical applications, they thereby enable others skilled in the art to utilize the invention and various embodiments with various modifications as are suited to the particular use contemplated. It is intended that the following claims and their equivalents define the scope of the invention.

1. A method for making a sensor arrangement for a user monitoring system, the method comprising:
providing an interconnected array of bases;
for each base:
applying a conductive material to a recessed portion in the base to form at least one conductive region;
coupling at least one sensor to the at least one conductive region;
coupling a conductive member to the at least one conductive region; and
dispensing an encapsulating material onto the base, thereby encapsulating the at least one sensor and at least a portion of the conductive member in the recessed portion.

2. The method of claim 1, wherein each base in the interconnected array of bases is formed from laser direct structuring.

3. The method of claim 1, wherein the conductive material comprises tin.

4. The method of claim 1, wherein the encapsulating material comprises resin.

5. The method of claim 4, wherein the resin is at least one of ultraviolet resin and epoxy resin.

6. The method of claim 1, wherein the at least one sensor comprises a temperature sensor.

7. The method of claim 1, wherein applying the conductive material to form the at least one conductive region comprises:
applying the conductive material to form a first conductive region; and
applying the conductive material to form a second conductive region,
wherein a first end of the at least one sensor is coupled to the first conductive region and a second end of the at least one sensor is coupled to the second conductive region.

8. The method of claim 7, wherein current flows into the at least one sensor along the first conductive region and current flows out from the at least one sensor along the second conductive region.

9. The method of claim 7, wherein the recessed portion includes a non-conductive region between the first conductive region and the second conductive region.

10. The method of claim 1, wherein a shape of the recessed portion is configured to increase contact between the encapsulating material and the base.

11. The method of claim 10, wherein the shape of the recessed portion comprises a central cavity and at least one peripheral cavity adjoining the central cavity.

12. The method of claim 11, wherein the at least one peripheral cavity includes a first peripheral cavity and a second peripheral cavity.

13. The method of claim 12, wherein the first peripheral cavity and the second peripheral cavity are opposite each other across the central cavity.

14. The method of claim 12, wherein the shape further includes a third peripheral cavity and a fourth peripheral cavity.

15. The method of claim 14, wherein the third peripheral cavity and the fourth peripheral cavity are opposite each other across the central cavity.

16. The method of claim 15, wherein:
the first peripheral cavity and the third peripheral cavity are on a first side of the central cavity; and
the second peripheral cavity and the fourth peripheral cavity are on a second side of the central cavity.

17. The method of claim 16, wherein:
the first peripheral cavity and the third peripheral cavity are spaced apart on the first side of the central cavity, and
the second peripheral cavity and the fourth peripheral cavity are spaced apart on the second side of the central cavity.

18. The method of claim 1, further comprising:
detaching the interconnected array of bases to form a plurality of bases.

19. The method of claim 18, further comprising:
bundling a first respective conductive member coupled to a first base from the interconnected array of bases and a second respective conductive member coupled to a second base from the interconnected array of bases to form the sensor arrangement.

20. The method of claim 19, wherein bundling the first respective conductive member and the second respective conductive member includes routing the first respective conductive member and the second respective conductive member into a predetermined shape.

21. The method of claim 20, wherein the predetermined shape includes a curved path.

22. The method of claim 21, wherein the curved path comprises a serpentine portion.

23. The method of claim 20, wherein the routing comprises routing the first respective conductive member and the second respective conductive member using a fixture, and wherein the fixture includes a slot having the predetermined shape.

24. The method of claim 19, further comprising:
encasing the sensor arrangement in a protective film.

25. The method of claim 24, wherein the protective film is polyurethane film.

26. The method of claim 1, further comprising:
coupling the sensor arrangement to a garment thereby forming the user monitoring system.

27. The method of claim 26, wherein the garment is configured to be placed on a foot of a user.

28. The method of claim 27, wherein the garment is a sock.

29. The method of claim 27, wherein the sensor arrangement is configured to measure temperature of at least a portion of the foot of the user.

30. A sensor arrangement for monitoring a user, the sensor arrangement comprising:
at least one base comprising a recessed portion having at least one conductive region; and
at least one conductive member coupled to the at least one base,
wherein at least one sensor is coupled to the at least one conductive region,
wherein a portion of the at least one conductive member in coupled to the at least one conductive region, and
wherein an encapsulating material encapsulates the at least one sensor and the portion of the at least one conductive member in the recessed portion of the at least one base.

31. The sensor arrangement of claim 30, wherein the at least one sensor comprises a temperature sensor.

32. The sensor arrangement of claim 30, wherein the at least one conductive region comprises a first conductive region and a second conductive region,
wherein a first end of the at least one sensor is coupled to the first conductive region and a second end of the at least one sensor is coupled to the second conductive region.

33. The sensor arrangement of claim 32, wherein current flows into the at least one sensor along the first conductive region and current flows out from the at least one sensor along the second conductive region.

34. The sensor arrangement of claim 32, wherein the recessed portion includes a non-conductive region between the first conductive region and the second conductive region.

35. The sensor arrangement of claim 30, wherein a shape of the recessed portion is configured to increase contact between the encapsulating material and the at least one base.

36. The sensor arrangement of claim 35, wherein the shape of the recessed portion comprises a central cavity and at least one peripheral cavity adjoining the central cavity.

37. The sensor arrangement of claim 36, wherein the at least one peripheral cavity includes a first peripheral cavity and a second peripheral cavity.

38. The sensor arrangement of claim 37, wherein the first peripheral cavity and the second peripheral cavity are opposite each other across the central cavity.

39. The sensor arrangement of claim 37, wherein the shape further includes a third peripheral cavity and a fourth peripheral cavity.

40. The sensor arrangement of claim 39, wherein the third peripheral cavity and the fourth peripheral cavity are opposite each other across the central cavity.

41. The sensor arrangement of claim 40, wherein:
the first peripheral cavity and the third peripheral cavity are on a first side of the central cavity; and
the second peripheral cavity and the fourth peripheral cavity are on a second side of the central cavity.

42. The sensor arrangement of claim 41, wherein:
the first peripheral cavity and the third peripheral cavity are spaced apart on the first side of the central cavity, and
the second peripheral cavity and the fourth peripheral cavity are spaced apart on the second side of the central cavity.

43. The sensor arrangement of claim 30, wherein the at least one conductive member comprises a plurality of conductive members.

44. The sensor arrangement of claim 43, wherein each conductive member of the plurality of conductive members is coupled to a respective base of the at least one base.

45. The sensor arrangement of claim 44, wherein each conductive member of the plurality of conductive members are bundled together to form the sensor arrangement.

46. A system comprising the sensor arrangement of claim 30, the system further including:
a garment coupled to the sensor arrangement and configured to be placed on a foot of a user.

47. The system of claim 46, wherein the system is configured to measure a temperature of the foot of the user.

48. The system of claim 46, further comprising:
a cover coupled to the garment, wherein the sensor arrangement is placed between the cover and the garment.

49. The system of claim 46, wherein the sensor arrangement is encased in a protective film.

50. The system of claim 49, wherein the protective film is polyurethane film.

51. The system of claim 46, wherein the garment is a sock.
